(19) United States
(12) Patent Application Publication
(10) Pub. No.: US 2008/0076957 A1
(43) Pub. Date: Mar. 27, 2008

(54) Method of producing europium-152 and uses therefor

(76) Inventor: Stuart Lee Adelman, Albuquerque, NM (US)

Correspondence Address: Stuart Lee Adelman, Ph.D, Suite 513, 12200 Academy Road NE, Albuquerque, NM, 87111, US

(21) Appl. No.: 11/527,288

(22) Filed: Sep. 26, 2006

(51) Int. Cl. A61N 5/00

(52) U.S. Cl. 600/8; 600/1

(57) ABSTRACT

Provided herein is a method of producing europium-152 as a radiotherapeutic source for external beam radiation therapy and brachytherapy using existing containment or capsule devices for the radionuclide. The method comprises irradiating a europium-151 enriched target with neutrons confined to a range of neutron energies effective to drive the reaction ¹⁵¹Eu(n,γ)¹⁵²Eu. Also provided are methods of treating a subject using external beam radiation therapy or brachytherapy using europium-152.

### BACKGROUND OF THE INVENTION

1. Field of the Invention

The present invention relates generally to the fields of nuclear physics and nuclear medicine. More specifically, the present invention relates to a method of producing europium-152 for use in external beam radiotherapy and brachytherapy.

2. Description of the Related Art

External beam radiotherapy (EBRT) or teletherapy is defined as the administration of ionizing radiation to human tissue, that is, a diseased organ of any sort or a neoplasm, malignant or benign, from a source of radiation located outside the patient's body. Any such source of radiation, whether machine generated, as in x-rays or linear accelerators, or radioactive isotopes which, during the course of their decay, emit gamma rays or beta particles. Today, most teletherapy devices in the world rely on gamma radiation to produce the therapeutic effect.

In the conventional isotope-teletherapy device which utilizes radioactive materials, i.e., radioisotopes or radionuclides, as their sources of radiation, the patient generally lies supine in a dedicated room, whereupon the teletherapy machine is mounted above him on a counterbalanced rotating mount, so that the beam of radiation is directed through various parts of the patient's skin, but focuses on the target organ or tumor some depth below the surface of the patient's skin. A high-intensity source of gamma radiation, generally several thousand curies, or several hundred trillion becquerels, of radioactivity is mounted within the teletherapy machine, such that the beam-intensity will deliver 100 to 200 rads (1 to 2 grays) per minute of absorbed dose to the target within the patient's body at a source-to-skin distance from 80 centimeters to 1 meter.

The ideal radioisotope will have a half-life many years long, emit gamma photons whose energy ranges from several hundred kilo-electron-volts (keV) to greater than one million electron-volts (MeV) and have a specific activity high enough that the entire radioactive mass will fit conveniently into a durable source capsule several centimeters in diameter and several centimeters high. The principal advantage of using radioisotopes as the generator of clinically useful radiation is that the energy of the gamma photons is fixed by natural law, as is the rate of emission of such gamma radiation.

Since the mid-twentieth century, when artificial radioisotopes became generally available, the principal isotope of choice for teletherapy has been the radioactive isotope “cobalt-60”. Currently, there are millions of high-activity cobalt-60 sources extant worldwide (1). Almost 10,000 of them in use in medical facilities, such as hospitals, free-standing clinics and cancer centers.

Cobalt-60 has numerous desirable characteristics for external beam radiotherapy. For example, cobalt-60 has a half-life of 5.27 years with a wide and plentiful distribution of ore available for refinement. It has a metal density of about 8 grams per cubic centimeter and possesses a simple chemistry which makes its conversion into a form useful for irradiation in a nuclear reactor both simple and straightforward. Cobalt-60 has a useful distribution of gamma energies, i.e, two gamma photons with every decay at 1.17 MeV and at 1.33 MeV.

However, cobalt-60 has several serious drawbacks for use as the ideal isotope for external beam radiotherapy. For example, with an activation cross-section (σ_{act}) of 20 barns, cobalt-60 has a small probability that an atom of naturally-occurring, stable cobalt-59 will be converted in a nuclear reactor to the radioactive form, cobalt-60 by the reaction ⁵⁹Co(n,γ)⁶⁰Co. A requirement for clinical usefulness dictates that no less than 5000 curies (Ci) (1.85×10¹⁴ becquerels (Bq)) of cobalt-60 be contained in a single capsule for placement into an external beam radiotherapy or teletherapy device with, therefore, a consequent requirement that clinically useful cobalt-60 be produced with a specific activity no less than 200 Ci·g⁻¹ (7.4×10¹² Bq·g⁻¹), contained in a single capsule whose inner diameter is generally less than 3 centimeters, so as to minimize the focal spot of the beam at the point where the beam intersects the subject's skin. Cobalt-60 must be irradiated for a period of 1 to 4 years even in high-flux reactors until an adequate specific activity of cobalt-60 is attained. This necessitates that an accurate prediction must be made of the demand for cobalt-60 years in the future to avoid either a shortage of cobalt-60 or a glut on the market.

However, improved techniques in lanthanide chemistry and chemical engineering has advanced to the point where the separation and purification of europium compounds has become routine. Also, the plasma enrichment process has reduced the difficulty and constructive impracticability of enriching the europium-151 precursor of europium-152 by several orders of magnitude. Europium-151 can be enriched to greater than 94% in adequate quantities. Furthermore, the availability of very high flux reactors for the production of medically significant isotopes has allowed the irradiation of large quantities of europium-151 as a practical technique. As such, enriched europium-152 sources, with a half-life approximately 2.5 times longer than cobalt-60, would be more advantageous for use in external beam radiation devices (1-5).

Thus, there is a recognized need in the art for a suitable substitute for cobalt-60 for use in isotope-teletherapy which overcomes the production and economic disadvantages associated therewith and for improved methods of producing europium-152 sources for medical use. Thus, the prior art is still deficient in the lack of using europium-152 in nuclear medicine. Specifically, the prior art is deficient in the lack of methods of activating europium-151 to produce europium-152 suitable for use in external beam radiation therapies or brachytherapies. The present invention fulfills this long-standing need and desire in the art.

### SUMMARY OF THE INVENTION

The present invention is directed to a method of producing europium-152. The method comprises irradiating a substantially isotopically pure europium-151 compound with neutrons having energies confined to a range of energies effective to substantially produce a europium-152 compound therefrom. A related method comprises a further step of encapsulating the substantially isotopically pure europium-151 compound within a capsule prior to the irradiating step.

The present invention is directed to a related method of producing europium-152 useful for external beam radiotherapy. The method comprises encapsulating a substantially isotopically pure europium-151 compound within a capsule adapted for use in an external beam radiotherapeutic device and irradiating the europium-151 compound inside the capsule with neutrons having an energy confined to a range of energies effective to substantially produce a europium-152 compound therefrom. A related method comprises a further step of operationally positioning the capsule within an external beam radiotherapy device. Further still the related method comprises the step of delivering a radiotherapeutic dose of gamma radiation emitted from the europium-152 in the capsule to a subject having diseased tissue; e.g., a cancer, to treat the subject.

The present invention is directed to another related method of producing europium-152 useful for brachytherapy. The method comprises enclosing a substantially isotopically pure europium-151 compound within a radioisotope seed adapted for use in an external beam radiotherapeutic device. The europium-151 compound inside the radioisotope seed is irradiated with neutrons having an energy confined to a range of energies effective to substantially produce a europium-152 compound therefrom. A related method comprises a further step of loading one or more of the europium-152 seeds or one or more needle(s) containing the seed(s) into an implantation device. Further still the related method comprises implanting the seed(s) or needle(s) containing the europium-152 compound into a subject having diseased tissue; e.g., a cancer, and delivering a radiotherapeutic dose of gamma radiation emitted from the europium-152 to the subject to treat the subject.

The present invention also is directed to a radiotherapeutic source of gamma radiation for external beam radiotherapy (EBRT). The radiotherapeutic source comprises a capsule suitable to contain a radioisotope and adapted for use in an external beam radiotherapy device and a europium-152 compound encapsulated therein and whose total activity is about 540 TBq to about 900 TBq at a radiotherapeutic dose rate useful for external beam radiotherapy.

The present invention also is directed to a related radiotherapeutic source of gamma radiation for brachytherapy. The radiotherapeutic source comprises one or more seeds or one or more needles containing the seed(s) suitable to contain a radioisotope and adapted for use in brachytherapy and a europium-152 compound contained within the seed(s) containing about 1 Tbq of activity effective to deliver a radiotherapeutic dose rate useful for brachytherapy.

The present invention is directed further to a method of treating diseased tissue; e.g., a cancer, in a subject using external beam radiotherapy. The method comprises directing gamma radiation emitted from the radiotherapeutic source for external beam radiotherapy described herein operationally positioned within an external beam radiotherapy device to the diseased tissue; e.g., a cancer, thereby treating the diseased tissue; e.g., a cancer, in the subject.

The present invention is directed further to a related method of method of treating diseased tissue; e.g., a cancer, in a subject using brachytherapy. The method comprises implanting the radiotherapeutic source for brachytherapy described herein into the subject proximate to the diseased tissue; e.g., the cancer such that gamma radiation emitted from said radiotherapeutic source is delivered to the diseased tissue; e.g., cancer, thereby treating the diseased tissue; e.g., the cancer in the subject.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention are briefly summarized. The above may be better understood by reference to certain embodiments thereof which are illustrated in the appended drawings. These drawings form a part of the specification. It is to be noted; however, that the appended drawings illustrate preferred embodiments of the invention and therefore are not to be considered limiting in their scope.

FIG. 1A depicts the activation cross-section for pure ¹⁵¹Eu in the reaction ¹⁵¹Eu(n,γ)¹⁵²Eu.

FIG. 1B depicts the burn-up cross section for the reaction ¹⁵²Eu (n,γ)¹⁵³Eu.

FIG. 2A depicts the total neutron cross section over the energy range 0.1≦Eₙ≦0.8 eV for the reaction ¹⁵¹Eu(n,γ)¹⁵²Eu.

FIG. 2B depicts the absorption cross section over the energy range 0.3≦Eₙ≦0.8 eV for the reaction ¹⁵²Eu(n,γ)¹⁵³Eu.

FIG. 3 depicts the dose rate in grays per hour at selected distances from the long axis of a 10 cm needle for radionuclides ⁶⁰Co, ¹⁹²Ir, ¹³⁷Cs, and ¹⁵²Eu.

### DETAILED DESCRIPTION OF THE INVENTION

In one embodiment of the present invention there is provided a method of producing europium-152, comprising irradiating a substantially isotopically pure europium-151 compound with neutrons having energies confined to a range of energies effective to substantially produce a europium-152 compound therefrom.

Further to this embodiment the method may comprise enclosing the substantially isotopically pure europium-151 compound within a capsule prior to the irradiating step. In one aspect of this further embodiment the capsule may be adapted for use in an external beam radiotherapy device (EBRT). In this aspect the europium-152 compound produced within the external beam radiotherapy capsule is a radiotherapeutic source of about 540 TBq to about 900 TBq. In another aspect of this further embodiment, the capsule is a seed adapted for use during brachytherapy. In this aspect the europium-152 compound produced within the brachytherapy seed is a radiotherapeutic source of about 1 Tbq.

In these embodiments the substantially isotopically pure europium-151 compound may be europium nitride. Also, the range of neutron energies is about 0.1 eV to about 0.8 eV. For example, the neutron energy may be about 0.46 eV. Furthermore, in these embodiments the specific activity of the europium-152 is about 3.5 terabequerels per gram to about 6 terabequerels per gram.

In a related embodiment the present invention provides a method of producing europium-152 useful for external beam radiotherapy, comprising encapsulating a substantially isotopically pure europium-151 compound within a capsule adapted for use in an external beam radiotherapeutic device; and irradiating the europium-151 compound inside the capsule with neutrons having an energy confined to a range of energies effective to substantially produce a europium-152 compound therefrom.

Further to this embodiment the method may comprise operationally positioning the capsule within an external beam radiotherapy device. Further still, the method may comprise delivering a radiotherapeutic dose of gamma radiation emitted from the europium-152 in the capsule to a subject having diseased tissue; e.g., a cancer to treat said subject. In these further embodiments the delivered radiotherapeutic dose is about 1.0 Gy min⁻¹ to about 5 Gy min⁻¹ at a source-to-skin-distance of about 80 centimeters to about 1 meter. For example, the delivered radiotherapeutic dose may be about 2.8 Gy min⁻¹ at a source-to-skin distance of about 1 meter.

In all embodiments the europium-152 compound may be a radiotherapeutic source of about 540 TBq to about 900 TBq. Also in all embodiments the neutron energies and the europium-151 compound are as described supra.

In another related embodiment the present invention provides a method of producing europium-152 useful for brachytherapy, comprising enclosing a substantially isotopically pure europium-151 compound within a radioisotope seed adapted for use in an external beam radiotherapeutic device; and irradiating the europium-151 compound inside the radioisotope seed with neutrons having an energy confined to a range of energies effective to substantially produce a europium-152 compound therefrom.

Further to this embodiment the method may comprise loading one or more of the europium-152 seeds or one or more needle(s) containing the seed(s) into an implantation device. Further still, the method may comprise implanting the seed(s) or needle(s) containing the europium-152 compound into a subject having diseased tissue; e.g., a cancer; and delivering a radiotherapeutic dose of gamma radiation emitted from the europium-152 to the subject to treat the subject. In these further embodiments the radiotherapeutic dose is about 8 grays per minute at an isodose line of about 1 centimeter.

In all of these embodiments the europium-152 compound may be a radiotherapeutic source of about 1 Tbq. Also in all embodiments the neutron energies and the europium-151 compound are as described supra.

In another embodiment of the present invention there is provided a radiotherapeutic source of gamma radiation for external beam radiotherapy (EBRT), comprising a capsule suitable to contain a radioisotope and adapted for use in an external beam radiotherapy device; and a europium-152 compound encapsulated therein and effective to deliver about 540 TBq to about 900 TBq at a radiotherapeutic dose rate for external beam radiotherapy.

In this embodiment the europium-152 compound may be europium-152 nitride. Also, in this embodiment the external beam radiotherapy radiotherapeutic dose rate is about 1.0 Gy min⁻¹ to about 5 Gy min⁻¹ at a source-to-skin-distance of about 80 centimeters to about 1 meter. For example, the radiotherapeutic dose rate may be about 2.8 Gy min⁻¹ at a source-to-skin distance of about 1 meter.

In a related embodiment the present invention provides a radiotherapeutic source of gamma radiation for brachytherapy, comprising one or more seeds or one or more needles containing the seed(s) suitable to contain a radioisotope and adapted for use in brachytherapy; and a europium-152 compound contained within the seed(s) containing about 1 Tbq and effective to deliver a radiotherapeutic dose rate for brachytherapy.

In this embodiment the europium-152 compound may be europium-152 nitride. Also, in this embodiment the radiotherapeutic dose rate may be about 8 Gy min⁻¹ at an isodose line of about 1 centimeter.

In yet another embodiment of the present invention there is provided a method of treating a diseased tissue; e.g., cancer, in a subject using external beam radiotherapy, comprising directing gamma radiation emitted from the radiotherapeutic source for external beam radiotherapy described herein operationally positioned within an external beam radiotherapy device to the diseased tissue; e.g., cancer, thereby treating the diseased tissue; e.g., cancer, in the subject.

In a related embodiment the present invention provides a method of treating diseased tissue; e.g., a cancer, in a subject using brachytherapy, comprising implanting the radiotherapeutic source for brachytherapy described herein into the subject proximate to the diseased tissue; e.g., the cancer, such that gamma radiation emitted from the radiotherapeutic source is delivered to the diseased tissue; e.g., the cancer, thereby treating the diseased tissue; e.g., the cancer, in the subject.

As used herein, the term, “a” or “an” may mean one or more. As used herein in the claim(s), when used in conjunction with the word “comprising”, the words “a” or “an” may mean one or more than one.

As used herein, the term “subject” refers to any target of the treatment.

The present invention provides methods of producing europium-152 from a europium-151-enriched compound, such as europium nitride (¹⁵²EuN), using flux optimization or spectrum tailoring. The europium-152 is produced in a format suitable as a replacement or substitute for cobalt-60 in external-beam radiotherapy (EBRT) and for iridium-92 in high-dose-rate brachytherapy (HDR). The method is significantly more cost effective than current methods of cobalt-60 production suitable for external beam radiotherapy and brachytherapy.

More particularly, highly enriched europium-151 nitride (¹⁵¹EuN) can be activated in a reactor with a significant flux of fast neutrons, by the reaction, ¹⁵¹Eu(n,γ)¹⁵²Eu with a probability, i.e., activation cross-section, σ_{act}, over 300 times that of the reaction, ⁵⁹Co(n,γ)⁶⁰Co. Europium-151 is irradiated or activated at neutron energies selected to produce europium-152 at high efficiency and minimize the “burn-out” of the europium-152 already created as the nuclear reaction progresses.

For example, the spectrum of possible neutron flux is contained within a range of neutron energies 0.1≦Eₙ≦0.8 eV and centered at 0.46 eV. Therefore, after accounting for regions of placement in the reactor, as well as neutron population and burn-up reactions, europium-152 can be irradiated to a clinically useful specific activity in less than 75 days. Preferably, europium-151 can be chemically converted to EuN, so that about 200 grams of elemental europium is activated to about 4.1 terabecquerels (TBq)/gram (110 Ci/g).

Furthermore, a suitable amount of europium-152, with a density of ρ=8.33 g·cm⁻³, can be produced in a clinically useful prefabricated capsule with identical dimensions to those currently used to enclose cobalt-60 metal. It is contemplated, therefore, that without alteration, the europium-152 capsule can be interchanged immediately with cobalt-60 source capsules in existing external beam radiotherapy machines or devices at an encapsulated total activity adequate for external beam radiotherapy. Preferably, about 740 TBq (20,000 Ci) of europium-152 can be produced in a standard-sized cobalt-60-type capsule with a specific activity from about 3.5TBq·g⁻¹ (94.6 Ci·g⁻¹) to about 6TBq·g⁻¹ (162 Ci·g⁻¹).

Thus, the present invention provides a method of external-beam radiotherapy. A subject in need of such treatment may receive radiation from a ¹⁵²europium nitride source enclosed or encapsulated in a capsule disposed in an external beam radiotherapy device. Encapsulated radiosources containing about 670 TBq (18,000 Ci) to about 815 TBq (22,000 Ci) of europium-152 can deliver a clinically acceptable absorbed dose rate of about 1.0 gray (Gy) per minute to about 5 grays per minute, preferably 1.5 grays per minute to about 2.8 grays per minute at a source-to-skin distance from about 80 cm to about 1 meter. Europium-152 emits clinically desirable gamma energies from 344 keV to 1.414 MeV, after beam hardening. Devices for external beam radiotherapy and the capsules containing the radionuclide are known and standard in the art. External beam radiotherapy is useful to treat diseased tissue(s); e.g., cancers, in a subject.

Alternatively, the europium-152 produced by the method described herein is suitable to replace iridium-192, or other radioisotopes; e.g., cesium-131, cesium-137, or ytterbium-169, as a radiotherapeutic source for brachytherapy. The present invention therefore also provides a method of brachytherapy. A subject in need of such treatment may receive radiation from brachytherapy seeds or needles comprising the europium-152 at linear isodose rates equal to those provided by standard iridium-192 seeds or needles. The brachytherapy seeds or needles containing two or more seeds are loaded into an implantation device and implanted at or delivered to a site of interest within or proximate to a tumor or diseased tissue; e.g., cancer, within a subject. Brachytherapy seeds and needles and devices and methods of delivery or implantation are known and standard in the art.

Preferably, a seed may contain a europium-152 nitride source of about 1.0 TBq (˜27 Ci). A brachytherapy needle may comprise two seeds and with an isodose line approximately 1 cm distant from the axis of the needle would, therefore, represent a dose rate of about 500Gy·h⁻¹ (˜8Gy·min⁻¹). It is contemplated that a dose of about 30 grays may be delivered during a 4 minute radiotherapeutic treatment.

The following examples are given for the purpose of illustrating various embodiments of the invention and are not meant to limit the present invention in any fashion.

### EXAMPLE 1

### Competing Resonance Absorption Peaks of ¹⁵¹Eu(n,γ)¹⁵²Eu and ¹⁵²Eu(n,γ)¹⁵³Eu

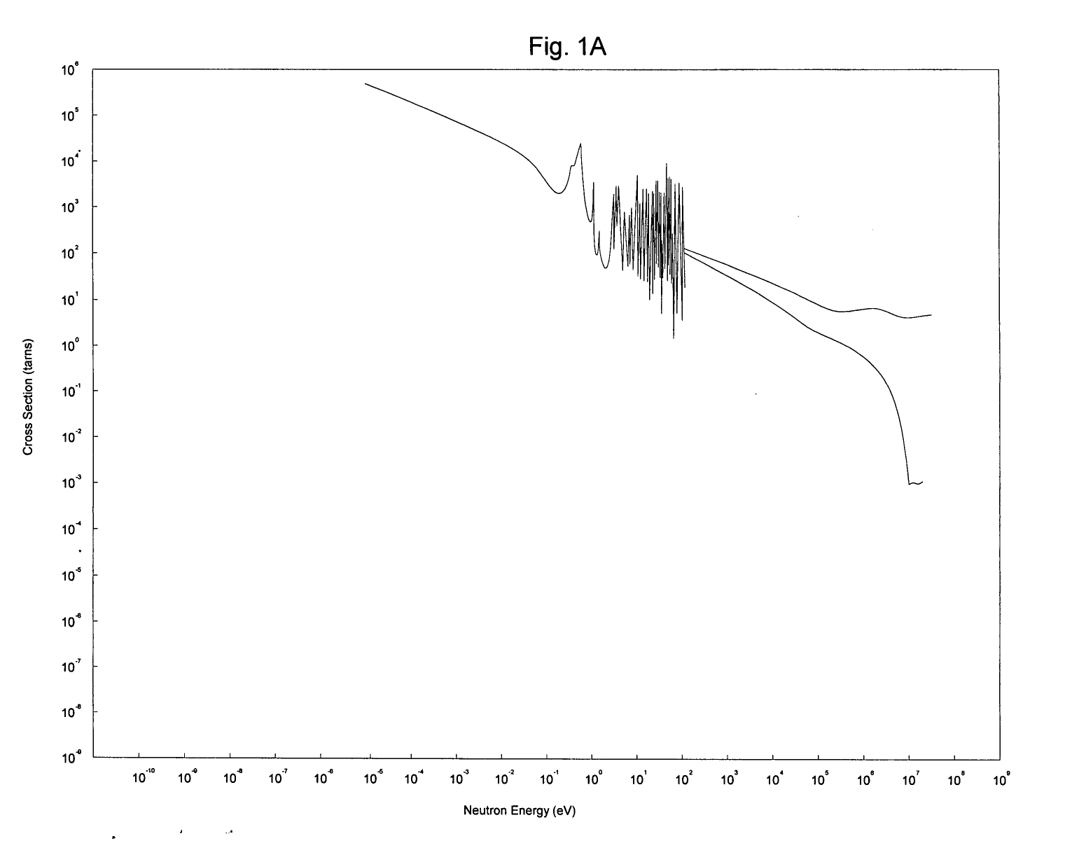

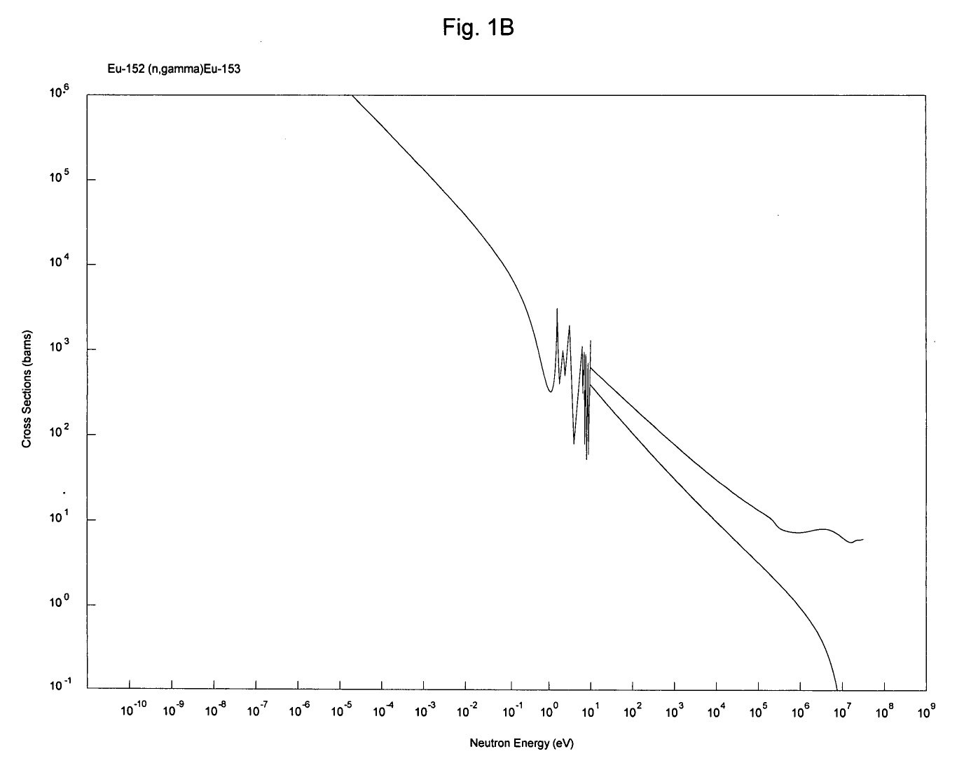

Europium-151, the target nuclide from which to produce europium-152, is remarkable, if not unique, because of its extraordinarily large activation cross-section for the reaction, ¹⁵¹Eu(n,γ)¹⁵²Eu, in the epithermal neutron region (FIG. 1A). That cross-section, as a function of neutron energy, σ_{act}(Eₙ), contains multiple resonance absorption peaks in the neutron energy range from about 0.125 eV to about 100 eV. Unfortunately, as the irradiation progresses, there exist strong competing resonances in the “burn-up” reaction, ¹⁵²Eu(n,γ)¹⁵³Eu (FIG. 1B), which are significant over the neutron spectrum for ˜0.025 eV ≦Eₙ≦0.11 eV and 0.9≦Eₙ≦˜9.0 eV. The existence of these very large, conflicting, resonance absorption peaks, one for the creation of the desired ¹⁵²Eu and the other for its burn-up to form ¹⁵³Eu, generates a situation in straightforward reactor physics which would seem to place an upper limit on the achievable specific activity of ¹⁵²Eu of only 2.25TBq·g⁻¹ (61 Ci·g⁻¹) compared to the theoretically limiting specific activity for ¹⁵²Eu of 6.5TBq g⁻¹ (176 Ci·g⁻¹).

### EXAMPLE 2

### Computer Simulations

The first step in the achievement of flux optimisation or spectrum tailoring following analytical calculations is the computer simulation of the irradiation. Such simulations have historically accounted for so many deterministic and statistical second- and third-order effects that one has been able, with a high level of confidence, to expect the outcome of an actual production run to approach the computed predictions very closely indeed. The cross section data for both for europium-151 and europium-152 was obtained from the Japanese Evaluated Nuclear Data Library (JENDL) (6-7).

### EXAMPLE 3

### Simulated Production of Europium-152 Using Spectrum Tailoring

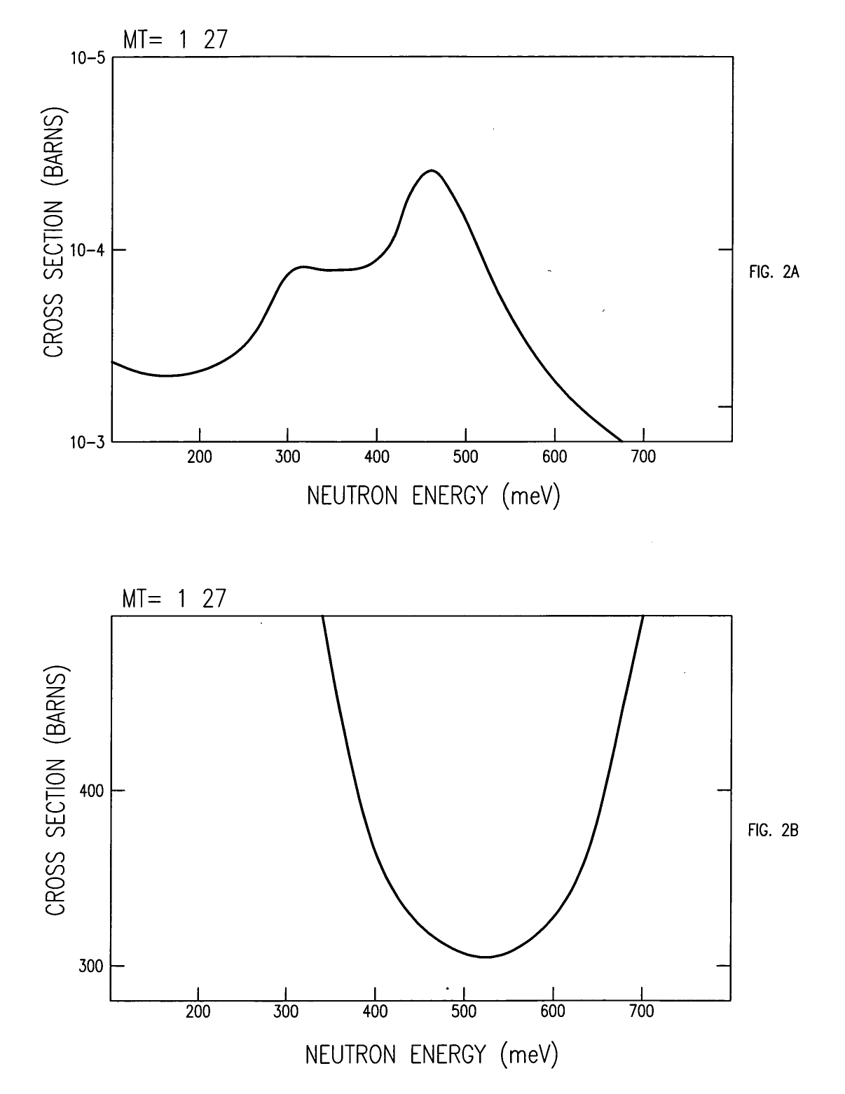

An inspection of FIGS. 2A-2B reveals that in the epithermal region, Eₙ≈0.46 eV, the activation cross section, σ_{act}(Eₙ), for the reaction, ¹⁵¹Eu (n,γ)¹⁵²Eu, has a resonance peak roughly equal to 2.6×10⁴ barns and that no such peak exists in the cross section for the competing reaction, where σ_{abs}≈320 barns for ¹⁵²Eu(n,γ)¹⁵³Eu. It is that neutron energy that was selected for centering a tailored spectrum.

Simulations, shown in Table I for the High Flux Isotope Reactor [HFIR] and the Fast Flux Test Reactor [FFTF] of the US Department of Energy, selected to tailor the neutron spectrum and centre it about the resonance peak at Eₙ=0.46 eV have shown a maximum total activity per gram of enriched ¹⁵¹EuN slightly greater than 5.8Tbq μg⁻¹ (˜156 Ci μg⁻¹). Table I includes the total activity and specific activity in the simulated production of ¹⁵²Eu by ¹⁵¹Eu(n,γ)¹⁵²Eu with spectrum tailoring where 0.1≦Eₙ≦0.8 eV. The target mass of europium-151 is 1 g of material where other stable nuclides, substantially gadolinium-152, samarium-152 and europium-153, account for 0.4 g of the original 1 g target mass.

TABLE I

| | Target | Irradiated | Product | Specific |
|---|---|---|---|---|
| | Mass | mass | Activity | Activity |
| | mᵢ (g) | mᵢf (g) | A (TBq) | A − [m_{f}(¹⁵¹Eu) + m_{f}(¹⁵²Eu)]⁻¹ |
| ¹⁵¹Eu | 1 | 0.108 | | |
| ¹⁵²Eu | 0 | 0.852 | 5.54 | 5.77 |

A=[Nσ_{act}φλ×(λ+σ_{abs}φ−σ_{act}φ)⁻¹]×([(exp−{σ_{act}φt})·(exp−t{λ+σ_{abs}φ})]

where σ_{act}=activation cross section for ¹⁵¹Eu(n,γ)¹⁵²Eu, and σ_{abs}=absorption cross section for the “burn-up” reaction, ¹⁵²Eu(n,γ)¹⁵³Eu.

These simulations also allow for self-shielding, under the presumption that the target material will be prefabricated into a source capsule containing 118 grams of 97% enriched ¹⁵¹EuN and, consequently, will contain just over 655TBq (˜18,000 Ci) of ¹⁵²Eu. In this particular simulation, over 99% of the target ¹⁵¹Eu is consumed and replaced by the product nuclide ¹⁵²Eu. The classical formulation that reveals specific activity in neutron activation reactions where burn-up must be taken into account, gives a similar value.

### EXAMPLE 4

### Europium-152 Capsule for EBRT

The europium-152 provides a source of about 540 TBq (˜14,600 Ci) to about 900 TBq (˜24,300 Ci) that may be contained within a capsule whose active volume would be less than 15.5 cm³, i.e., 2.7 cm inner diameter×2.7 cm in height, which is essentially identical to those used in existing cobalt-60 external beam therapy irradiators. Such capsules would deliver doses roughly equivalent to activities of 260 TBq (˜7000 Ci)≦x≦340 TBq (˜9200 Ci) of cobalt-60 with dose rates of 2≦x≦5Gy min⁻¹ at 80 cm≦x≦1 m source-to-skin-distance (SSD). Because a prefabricated source capsule is loaded with the stable target nuclide and irradiated as a unit, the need to manipulate many petabecquerels (hundreds of kilocuries) of amorphous radioisotope is obviated and the very great cost of a dedicated ultra-high-activity radiochemical laboratory can be avoided.

The fabrication of petabecquerel sources of europium-152, whose capsules are fully and directly interchangeable with the cobalt-60 capsules now extant, is entirely dependent upon the achievement of activities somewhere between 3.5TBq·g⁻¹ and 6TBq·g⁻¹ (94.6 Ci g⁻¹ and 162 Ci·g⁻¹) of target material. Such activities are practicable only by the use of flux optimisation. Moreover, those specific activities are quite impossible to achieve unless enriched target material is used, because europium-151 has a natural abundance of only 47.70%. The balance is comprised uniquely of europium-153.

Until the advent of the Plasma Enrichment Process (PEP) to concentrate very large quantities, e.g., tens of kilograms, of the stable, target isotope, europium-151 to ≧95% isotopic purity, the cost of such enrichment has been prohibitive, i.e., about $7.00 per milligram for 97% enrichment, or ˜$825,000 per source. Using the Plasma Enrichment Process, the cost of an encapsulated source of about 900 TBq (19,000 Ci) generating ˜2.8 Gy·min⁻¹ at 1 m, as in the capsule described herein, would cost less than $5000 per gray per minute at one metre.

Of course, the half-life of europium-152 is 2.5 times longer than that of cobalt-60, so that a source of europium-152 will be clinically useful for a significantly longer time than one of cobalt-60. Consequently, the cost of a europium-152 installation must be lessened by a factor of approximately 0.4, reflecting europium's greater longevity. Furthermore, some of europium's gamma energies will remain clinically useful even after scattering from the irradiator collimation. When both those factors are included, the real cost to a hospital of buying a europium-152 source, instead of a source using cobalt-60, that is, the price to be amortised by the hospital, would be decreased still further to ˜$000 per gray per minute at a metre, or ˜13% that of cobalt-60,

### EXAMPLE 5

### Europium-152 Capsule for Brachytherapy

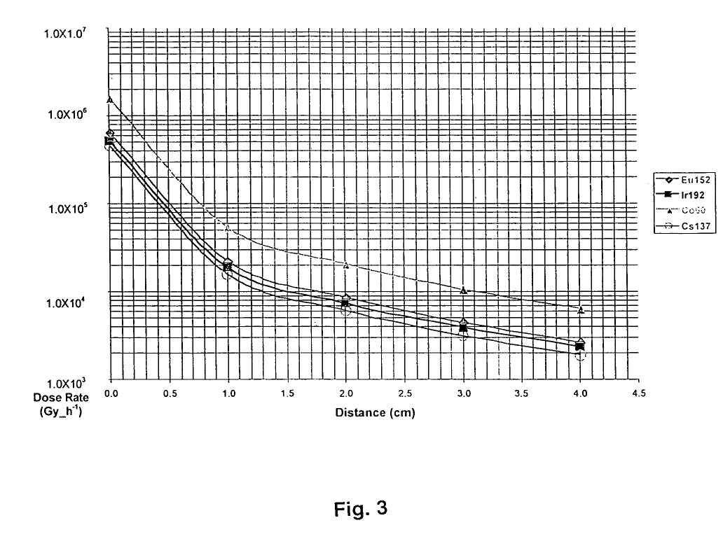

Ultra-high specific activity europium-152 also is effective for high-dose-rate (HDR) or pulsed-dose-rate (PDR) brachytherapy. For example, a typical needle used in breast brachytherapy following lumpectomy generally contains two or more radioactive “seeds”, cylindrical in shape, with dimensions of a 0.3 cm inner diameter by 0.3 cm length; i.e. volume, V, ˜0.02 cm³. Using europium nitride (EuN) at a density, ρ≈8.33 g·cm⁻³, such a seed could be made to contain ˜0.16 g of europium-152 or about 1.0 TBq (˜27 Ci). An isodose line approximately 1 cm distant from the axis of the needle would therefore represent a dose rate of about 500Gy·h⁻¹ (˜8Gy·min⁻¹) (FIG. 3). Consequently, a typical desired dose of 30 grays might be delivered in ˜4 min, an unusually high rate for standard high-dose-rate or pulsed-dose-rate treatment plans, superior even to those using far more costly, high-specific activity iridium-192.

Even seeds containing the novel medical nuclide cesium-131, are notably expensive when used for breast brachytherapy owing to a very short half-life, t_{1/2}=9.7 days, and the consequent need for frequent renewal. The availability of such very short-lived radionuclides as cesium-131 or palladium-103 becomes important only when, owing to difficulties in surgical access to a tumor, it becomes clinically desirable to leave the implanted seeds permanently in situ.

The following references are cited herein.
1. Adelman, S. L. Med. Phys. 23 (1996) 1443.
2. Adelman, S. L. Jpn. J. Appl. Phys. 37 (1998) L1277.
3. Adelman, S. L. Jpn. J. Appl. Phys. 38 (1999) L1505.
4. Adelman, S. L. Recent Research Developments in Applied Chemistry, ed. S. G. Pandalai (Transworld Research Network, Trivandrum, India and Stevenage, England, 2002) p. 67.
5. Adelman, S. L. and Bowden-Adelman, J.C. Proc. 8^{th} Int. Symp. Synthesis and Applications of Isotopes and Isotopically Labelled Compounds, eds. D. Dean, C. Filer, J. Wiley and Sons, Chichester, 2004) in press.
6. Adelman, et al. Ann. Conf. of UK Group, Int. Isotope Soc., Hinxton, Cambridge, England, October 2003.
7. Shibata, et al. J. Nucl. Sci. Technol. 39, 1125 (2002).

Any publications or patents mentioned in this specification are indicative of the levels of those skilled in the art to which the invention pertains. Further, these publications are incorporated by reference herein to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of producing europium-152, comprising:
irradiating a substantially isotopically pure europium-151 compound with neutrons having energies confined to a range of energies effective to substantially produce a europium-152 compound therefrom.

2. The method of claim 1, further comprising:
encapsulating said substantially isotopically pure europium-151 compound within a capsule prior to the irradiating step.

3. The method of claim 2, wherein said capsule is adapted for use in an external beam radiotherapy device.

4. The method of claim 3, wherein the europium-152 compound produced within said external beam radiotherapy device capsule is a radiotherapeutic source of about 540 TBq to about 900 TBq.

5. The method of claim 2, wherein said capsule is a seed adapted for use during brachytherapy.

6. The method of claim 5, wherein said europium-152 compound produced within said brachytherapy seed is a radiotherapeutic source of about 1 Tbq.

7. The method of claim 1, wherein said substantially isotopically pure europium-151 compound is europium nitride.

8. The method of claim 1, wherein said range of neutron energies is about 0.1 eV to about 0.8 eV.

9. The method of claim 8, wherein said neutron energy is about 0.46 eV.

10. The method of claim 1, wherein the specific activity of said europium-152 is about 5 terabequerels per gram to about 6 terabequerels per gram.

11. A method of producing europium-152 useful for external beam radiotherapy, comprising:
encapsulating a substantially isotopically pure europium-151 compound within a capsule adapted for use in an external beam radiotherapeutic device; and
irradiating said europium-151 compound inside the capsule with neutrons having an energy confined to a range of energies effective to substantially produce a europium-152 compound therefrom.

12. The method of claim 11, further comprising:
operationally positioning said capsule within an external beam radiotherapy device.

13. The method of claim 12, further comprising:
delivering a radiotherapeutic dose of gamma radiation emitted from the europium-152 in said capsule to a subject having diseased tissue; e.g., a cancer, to treat said subject.

14. The method of claim 13, wherein said delivered radiotherapeutic dose is about 1.0 Gy min⁻¹ to about 5 Gy min⁻¹ at a source-to-skin-distance of about 80 centimeters to about 1 meter.

15. The method of claim 14, wherein said delivered radiotherapeutic dose is about 2.8 Gy min⁻¹ at a source-to-skin distance of about 1 meter.

16. The method of claim 11, wherein said range of neutron energies is about 0.1 eV to about 0.8 eV.

17. The method of claim 16, wherein said neutron energy is about 0.46 eV.

18. The method of claim 11, wherein said substantially isotopically pure europium-151 compound is europium nitride.

19. The method of claim 11, wherein said europium-152 compound is a radiotherapeutic source of about 540 TBq to about 900 TBq.

20. A radiotherapeutic source of gamma radiation for external beam radiotherapy, comprising:
a capsule suitable to contain a radioisotope and adapted for use in an external beam radiotherapy device; and
a europium-152 compound encapsulated therein and effective to deliver about 540 TBq to about 900 TBq at a radiotherapeutic dose rate useful for external beam radiotherapy.

21. The radiotherapeutic source of claim 20, wherein said europium-152 compound is europium-152 nitride.

22. The radiotherapeutic source of claim 20, wherein said external beam radiotherapy device radiotherapeutic dose rate is about 1.0 Gy min⁻¹ to about 5 Gy min⁻¹ at a source-to-skin-distance of about 80 centimeters to about 1 meter.

23. The radiotherapeutic source of claim 22, wherein said radiotherapeutic dose rate is about 2.8 Gy min⁻¹ at a source-to-skin distance of about 1 meter.

24. A method of treating diseased tissue; e.g., a cancer, in a subject using external beam radiotherapy, comprising:
directing gamma radiation emitted from the radiotherapeutic source of claim 20 operationally positioned within an external beam radiotherapy device to the diseased tissue; e.g., cancer, thereby treating the diseased tissue; e.g., cancer, in the subject

25. A radiotherapeutic source of gamma radiation for brachytherapy, comprising:
one or more seeds or one or more needles containing said seed(s) suitable to contain a radioisotope and adapted for use in brachytherapy; and
a europium-152 compound contained within said seed(s) and effective to deliver a radiotherapeutic dose rate of about 8 Gy·min⁻¹ at an isodose line of about 1 cm: a dose rate useful for brachytherapy.

26. The radiotherapeutic source of claim 25, wherein said europium-152 compound is europium-152 nitride.
